(19) United States
(12) Patent Application Publication
(10) Pub. No.: US 2022/0117929 A1
(43) Pub. Date: Apr. 21, 2022

(54) METHOD FOR PREVENTING OR TREATING HIGH-GRADE SEROUS CARCINOMA

(71) Applicant: Buddhist Tzu Chi Medical Foundation, Hualien City (TW)

(72) Inventor: Dah-Ching Ding, Hualien City (TW)

(21) Appl. No.: 17/075,877

(22) Filed: Oct. 21, 2020

(51) Int. Cl. A61K 31/343; A61K 31/337; A61K 33/243; A61K 31/7068; A61K 31/704

(52) U.S. Cl. CPC A61K 31/343; A61K 31/337; A61K 31/704; A61K 31/7068; A61K 33/243

(57) ABSTRACT

Provided is a method of preventing or treating high-grade serous carcinoma in a subject in need thereof, including administering to the subject a composition including an effective amount of n-butylphthalide.

### BACKGROUND OF THE INVENTION

### 1. Technical Field

The present disclosure relates to a method for preventing or treating high-grade serous carcinoma by administering a composition comprising an effective amount of butylidenephthalide to a subject in need thereof.

### 2. Description of Associated Art

Cancers remain the leading causes of death worldwide, accounting for 9.5 million deaths in 2018. In 2018 alone, a total of 18 million new cancer cases were diagnosed. While ovarian cancer is not the most frequent, it is the deadliest form of gynecological malignancy in woman. While the survival rates for a number of solid tumors have improved significantly in the last 50 years with the advance in many aspects of medicine and technology, however, the 5-year overall survival from ovarian cancer remains virtually unchanged since about 1980. According to the most recent figures published by the Surveillance, Epidemiology and End Results (SEER) program of the American National Cancer Institute (NCI), the current 5-year survival rate (2010 to 2016) in the US is approximately 48.6%, with the death rate at 6.9 per 100,000 women per year.

Ovarian cancer comprises many different subtypes and is classified according to the histological origin of the transformation of cancer cells. About 90% of ovarian tumors are deemed to have occurred through the transformation of epithelial cells as opposed to those originating from germ cells or sex-cord-stromal tissues, which is named epithelial ovarian cancers (EOC). EOC is further divided into four well-defined histological subtypes referred to as serous, mucinous, clear-cell and endometrioid. Additional level of stratification of these subtypes can further take into account of tumor grade, based on the apparent degree of cytological aberration. Therefore, high-grade and low-grade serous carcinomas of the ovary are now considered to be two entirely different neoplasms, with distinct modes of carcinogenesis, molecular-genetic features and sites of origin, although they share some similarity in histological appearance and terminology. While the majority of cases observed clinically belongs to one of the four major histological types, a number of rarer types have been observed. These include malignant transitional cell tumors, also named Brenner tumor, as well as cases of mixed type and undifferentiated carcinoma.

Specifically, high-grade serous carcinoma (HGSC) is a type of tumor that arises from the serous epithelial layer in the abdominopelvic cavity and is mainly found in the ovary that includes serous tubal intraepithelial carcinoma, peritoneum serous carcinoma, fallopian tube serous carcinoma, uterine corpus serous carcinoma and cervix serous carcinoma. Although originally thought to arise from the squamous epithelial cell layer covering the ovary, HGSC of the ovary is now thought to originate in the fallopian tube epithelium. HGSC makes up the majority of ovarian cancer cases and is much more invasive than low-grade serous carcinoma (LGSC) with a higher fatality rate. HGSC, therefore, is distinct from LGSC, which arises from ovarian tissue and is less aggressive where tumors are localized to the ovary.

The current treatment for ovarian cancer generally involves debulking surgery and adjuvant chemotherapy with platinum and paclitaxel. Irrespective of this treatment, the recurrence rate of ovarian cancer remains high. Resistance to the current chemotherapeutic drugs may develop after multiline chemotherapies. In addition, the toxicity of these chemotherapeutic drugs is so high that some patients are precluded from completing the therapy. In addition, the treatment for ovarian cancer remains general without distinguishing the actual subtype of the ovarian cancer. Therefore, considering the malignancy of high-grade serous tumors and the insufficient therapeutic approaches available, developing a new drug or adjuvant to increase the efficacy of currently available drugs is highly needed.

### SUMMARY OF THE INVENTION

In view of the foregoing, the present disclosure provides a method for preventing or treating high-grade serous carcinoma in a subject in need thereof, comprising administering to the subject a composition comprising an effective amount of n-butylidenephthalide and a carrier thereof.

In an embodiment, the method of the present disclosure is provided with at least one additional treatment for high-grade serous carcinoma, such as surgery, chemotherapy, radiation therapy, targeted therapy, biological therapy or immunotherapy. In an embodiment, the chemotherapy is an adjuvant chemotherapy comprising administering to the subject an adjuvant chemotherapy drug selected from the group consisting of paclitaxel, carboplatin, cisplatin, gemcitabine, topotecan, and liposomal doxorubicin.

In an embodiment, the high-grade serous carcinoma to be treated by the present disclosure is a carcinoma that originates from the serous epithelial layer in abdominopelvic cavity. In another embodiment, the high-grade serous carcinoma is high-grade serous ovarian carcinoma.

In a further embodiment, the high-grade serous carcinoma is selected from the group consisting of peritoneum serous carcinoma, fallopian tube serous carcinoma and uterine corpus serous carcinoma.

In an embodiment, the n-butylidenephthalide for prevention or treatment in the present disclosure is purified from natural extracts of plants. In another embodiment, the n-butylidenephthalide is purified from natural extracts of Angelica sinensis. In a further embodiment, the n-butylidenephthalide is synthetically synthesized.

In the present disclosure, the prevention or treatment of the high-grade serous carcinoma comprises inducing apoptosis of cancer stem cells of the high-grade serous carcinoma. In an embodiment, the induction of apoptosis comprises increasing a protein level of at least one of cleaved caspase 3, cleaved caspase 7, cleaved caspase 8 or cleaved caspase 9.

In the present disclosure, the prevention or treatment of the high-grade serous carcinoma comprises inhibition of proliferation of cancer stem cells of high-grade serous carcinoma. In another embodiment, the prevention or treatment of the high-grade serous carcinoma comprises inhibition of invasion of cancer stem cells of the high-grade serous carcinoma. In a further embodiment, the prevention or treatment of the high-grade serous carcinoma comprises inhibition of migration of cancer stem cells of the high-grade serous carcinoma.

In an embodiment of the present disclosure, the n-butylphthalide is provided at a dosage of from about 10 mg/kg to about 500 mg/kg per day, for example, with a lower limit of 10 mg/ml, 25 mg/ml, 50 mg/ml, 75 mg/ml, 100 mg/ml or 150 mg/ml, and with an upper limit of 500 mg/ml, 400 mg/ml, 300 mg/ml, 250 mg/ml or 200 mg/ml. In an embodiment, the n-butylphthalide is provided at a dosage of from about 50 mg/kg to about 250 mg/kg per day, such as 50 mg/ml, 75 mg/ml, 100 mg/ml, 125 mg/ml, 150 mg/ml, 175 mg/ml, 200 mg/ml, 225 mg/ml and 250 mg/ml.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A to 1C show the characteristics of kuramochi cells with cancer stem cell (CSC) marker. FIG. 1A shows the expression of ALDH, a CSC marker, identified using the Aldefluor assay and measured by flow cytometry, in kuramochi cells. Kuramochi-ctrl-1: Kuramochi cell line without sorting. Kuramochi-ALDH1-1: cancer stem cell (ALDH⁺) of kuramochi cell line. Data are expressed as dot plots, and the percentage of ALDH⁺ cells in the total population are found to be 0.6% in Kuramoni-ctrl-1 cells and 3.6% in Kuramochi-ALDH1-1 cells, respectively. P5: a portion of cells with ALDH expression; P6: a portion of cells without ALDH expression. FIG. 1B shows the morphology of ALDH⁺ and ALDH⁻ kuramochi cells. FIG. 1C shows the proliferation of ALDH⁺ and ALDH⁻ kuramochi cells. The difference between the cell proliferation in ALDH⁺ and ALDH⁻ kuramochi cells is statistically different on day 5 and day 7. **p<0.01; ***p<0.001.

FIGS. 2A and 2B show the half-maximal inhibitory concentration (IC50) of BP after adding different concentrations of BP for 48 h. FIG. 2A shows the result using ordinary kuramochi cells. 2×10³ cells/cm² of ordinary kuramochi cells were plated in each well of 96-well plates and treated with different concentrations of BP (0, 15, 30, 60, 120 and 240 μg/ml). After 48 h of culture, the IC50 of BP for the ordinary kuramochi cells was calculated as 206.5 g/ml. FIG. 2B shows the result using ALDH⁺ kuramochi cells. The IC50 of BP (used at 0, 12.5, 25, 50, 100, 200, and 400 μg/ml) for 48 h in ALDH⁺ kuramochi cells (2×10³ cells/cm²) was calculated as 317.2 μg/ml.

FIGS. 3A to 3D show the decreased migration and invasion of ALDH⁺ kuramochi cells after treatment with BP. FIG. 3A shows the result of migration assays in ALDH⁺ kuramochi cells (5×10⁴ cells) with 200 μg/ml of BP or without the BP treatment (Ctrl) for 48 hours. FIG. 3B shows the result of migration assays in ALDH⁺ kuramochi cells (5×10⁴ cells) with 100 g/ml of BP or without the BP treatment for 48 hours. FIG. 3C shows the result of invasion assay in ALDH⁺ kuramochi cells (5×10⁴ cells) with 200 μg/ml of BP or without the treatment for 24 hours. FIG. 3D shows the result of invasion assay in ALDH⁺ kuramochi cells (5×10⁴ cells) with 100 μg/ml of BP or without the treatment for 24 hours. The upper panel of images were the representative images of the experiments. Ctrl: control. **p<0.01, ***p<0.001.

FIGS. 4A and 4B show the inhibition of ALDH⁺ kuramochi cell proliferation by BP via apoptosis. FIG. 4A shows the result of TUNEL assay of ALDH⁺ kuramochi cells with or without BP treatment (BP: 200 μg for 48 hours). Scale bar=100 m. FIG. 4B shows the histograms of quantification of TUNEL⁺ cells in both groups. Ctrl: control. DAPI: 4′,6-diamidino-2-phenylindole. **p<0.01.

FIGS. 5A to 5E show the activation of apoptosis signaling pathway by BP. After BP treatment (100 μg or 200 μg, 48 hours), Western blots show that protein levels of cleaved caspase 9 (FIG. 5A), cleaved caspase 8 (FIG. 5B), cleaved caspase 7 (FIG. 5C), and cleaved caspase 3 (FIG. 5D) increased in ALDH⁺ kuramochi cells. FIG. 5E shows the histogram quantification of the protein expression of cleaved caspase 3 (n=3). ***p<0.001. All cropped blots were run under the same experimental conditions. The numbers below each blot revealed the relative quantification (R.Q.) to beta-actin.

FIGS. 6A to 6B show the effects of BP on cisplatin and paclitaxel treatment in ALDH⁺ kuramochi cells. FIG. 6A shows the result of treating ALDH⁺ kuramochi cells with cisplatin (0, 5 and 10 μM) with or without additional BP (25 μg/ml). All experiments were conducted in triplicate. FIG. 6B shows the result of treating ALDH⁺ kuramochi cells with paclitaxel (0, 10, or 50 nM) with or without additional BP (25 μg/ml). All experiments were conducted in triplicate. *p<0.05; **p<0.01.

FIGS. 7A to 7E show the inhibition of xenograft tumor growth by BP via apoptosis. FIG. 7A shows the tumor volume with or without BP treatment (100 or 200 mg/kg for 5 days). Cells (1×10⁶) were injected subcutaneously into the backs of NOD-SCID mice. Tumor growth curves over 26 days are shown with vehicle control and BP treatment (100 or 200 mg/kg for 5 days). The mean relative tumor volumes are shown. ***p<0.001. Vit K: vitamin K, administered in the control group without BP treatment, as vitamin K was used to dissolve BP in the BP treatment group. FIG. 7B shows the hematoxylin and eosin staining of tumor tissue with or without BP treatment. Scale bar=100 m. FIG. 7C shows the TUNEL assay of tumor tissue with or without BP treatment. Scale bar=100 m. FIG. 7D shows the histogram quantification of TUNEL⁺ cells in both groups. ***p<0.001. FIG. 7E shows the increased protein levels of cleaved caspase 3, caspase 9, and caspase 7 in ALDH⁺ kuramochi xenograft. All cropped blots were run under the same experimental conditions. The numbers below each blot revealed the relative quantification to tubulin.

### DETAILED DESCRIPTION OF THE EMBODIMENTS

The present disclosure provides a use of n-butylidenephthalide (BP) in the manufacture of a medicament for preventing or treating of high-grade serous carcinoma (HGSC). The use of BP in treating HGSC comprises administering to a subject in need thereof a composition comprising an effective amount of BP, a metabolic precursor of BP, a pharmaceutically acceptable salt of a metabolic precursor of BP, a pharmaceutically acceptable ester of a metabolic precursor of BP, or combinations thereof.

BP is an active ingredient in the chlorinated layer of Angelicae sinensis, which is a common Chinese herbal medicine used for the treatment of cough, headache, and angina and to strengthen muscles. Therefore, BP used in this disclosure can be purified from the acetone extract, chloroform extract or hexane extract of Angelicae sinensis. Alternatively, BP can also be synthesized synthetically.

HGSC refers to tumors that arise from the serous epithelial layer in the abdominopelvic cavity. HGSC is found in the ovary, and is named as high-grade serous ovarian cancer (HGSOC). HGSC includes other carcinoma found in the pelvic, including serous tubal intraepithelial carcinoma, peritoneum serous carcinoma, fallopian tube serous carcinoma, uterine corpus serous carcinoma and cervix serous carcinoma. HGSC makes up the majority of ovarian cancer cases and is much more invasive with a higher fatality rate than low-grade serous carcinoma (LGSC). HGSC, therefore, is distinct from LGSC, which arises from ovarian tissue and is less aggressive where tumors are localized to the ovary.

Tumors are composed of cells with varying degrees of malignancy. Tumor development is mediated by specialized, pluripotent, and self-proliferating cells that have tumorigenic properties and are known as cancer stem cells (CSCs). The presence and numbers of CSCs largely determine the tumor aggressiveness, resistance to therapy, and disease relapse of cancer. However, the CSCs are often resistant to traditional treatment. Thus, effective cancer therapy is needed to specifically target CSCs. Stem cells of HGSOC can be identified and isolated for study using aldehyde dehydrogenase activity (ALDH) as a marker. Other CSC markers in ovarian cancer include cluster of differentiation 24 (CD24), CD44, CD117, CD133, and receptor tyrosine kinase like orphan receptor 1 (ROR1).

The present disclosure provides a method for preventing or treating HGSC comprising administering an effective amount of BP to a subject in need thereof, after finding that BP significantly inhibits HGSOC CSC proliferation both in vitro and in vivo. As disclosed herein, BP also inhibits HGSOC CSC migration and invasion. Furthermore, BP treatment results in the cell death of HGSOC CSCs via activation of the apoptosis signaling pathway. BP also increases the toxicity of the chemotherapeutic drugs, cisplatin and paclitaxel. Therefore, BP can be used in combination with other cancer therapies to increase the effectiveness of the cancer treatment. In mice, BP treatment inhibits the tumor growth. For example, BP induces apoptotic pathway in tumor cells in mice and leads to lowered cell viabilities of the tumors.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected in this disclosure, and in this case, the meaning of the selected terms will be described in detail in the descriptions of the present disclosure. Thus, the terms used herein have to be defined based on the meaning of the terms together with the descriptions throughout the specification.

Also, when a part “includes” or “comprises” a component or a step, unless there is a particular description contrary thereto, the part can further include other components or other steps, not excluding the others.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

The phrase “an effective amount” refers to the amount of an active ingredient that is required to result in a reduction, inhibition or prevention of the disorder, abnormality or symptom in the individual. An effective amount will vary, as recognized by those skilled in the art, depending on routes of administration, excipient usage, and the possibility of co-usage with other therapeutic treatment.

The term “individual” as used herein is interchangeable with “subject” and includes a single biological organism, including, but not limited to, animals such as vertebrates, mammals or human beings.

The term “individual in need of the treatment” refers to a subject expressing or suffering from one or more symptoms related to HGSC. An appropriately qualified person or physician is able to identify such an individual in need of treatment using standard testing protocols or guidelines. The same testing protocols or guidelines may also be used to determine whether there is improvement to the individual's disorders or symptoms, or determine the most effective dose of HGSC to be administered to an individual in need of the treatment.

The term “improvement” as used herein refers to prevention or reduction in the severity or frequency, to whatever extent, of one or more of the symptoms or abnormalities expressed by the individual diagnosed with HGSC. The improvement is either observed by the individual taking the treatment themselves or by another person.

Different examples have been used to illustrate the present disclosure. The examples below should not be taken as a limit to the scope of the present disclosure.

Statistical analysis is carried out in the examples to present data as the mean±SD of at least three independent experiments. The Mann-Whitney U test was used to compare two independent variables, and one-way ANOVA was used to compare three independent variables. Statistical analysis was performed using GraphPad Prism 6 (La Jolla, Calif., USA). A p value less than 0.05 is considered to have a significant difference.

### EXAMPLE

Exemplary embodiments of the present disclosure are further described in the following examples, which do not limit the scope of the present disclosure.

### Example 1: Identification and Characterization of HGSC Cancer Stem Cells in Kuramochi Cells

Kuramochi cells is a high-grade serous ovarian carcinoma (HGSOC) cell line purchased from Japan Cell Bank. Gene expressions of this cell line was previously confirmed to be mimicking HGSOC (Domcke, S.; Sinha, R.; Levine, D. A.; Sander, C.; Schultz, N. Nat. Commun. 2013, 4, 2126). The cell line was maintained in Dulbecco's Modified Eagle Medium (DMEM; Sigma, St. Louis, Mo., USA) supplemented with 10% fetal bovine serum (FBS), 0.1% non-essential amino acids (NEAA), 2 mM L-glutamine, and 1% penicillin-streptomycin. The cells were incubated at 37° C. with 5% CO₂.

To evaluate and characterize the kuramochi cells, the expression of the cancer stem cell (CSC) marker, ALDH, was assessed using flow cytometry. Specifically, fluorescence-activated cell sorting (FACS) was used to isolate ALDH⁺ cells from the kuramochi cell line. Then, the Aldefluor assay kit (Stem Cell Technologies, Cambridge, Mass., USA) was used to determine the ALDH activity. The activated Aldefluor reagent was used to trypsinized and incubated with cells for 50 min at 37° C. Cells were incubated with the inhibitor, N,N-diethylaminobenzaldehyde (DEAB), as control cells to identify for ALDH⁺ and ALDH⁻ cell populations. BD FACSVerse flow cytometer (BD Biosciences, San Jose, Calif., USA) was used to examine and analyze all the stained cells. Then, BD FACSAria Fusion flow cytometer (BD Biosciences, San Jose, Calif., USA) was used for sorting ALDH⁺ cells. After sorting, ALDH⁺ and ALDH⁻ cells were propagated using the above culture medium for no more than five passages. It was found that the percentage of ALDH⁺ cells was more than 80% within 5 passages.

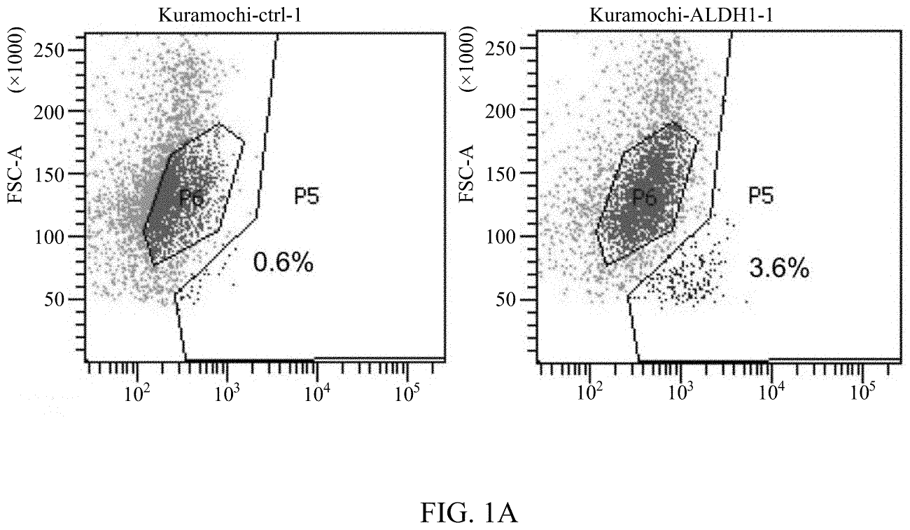

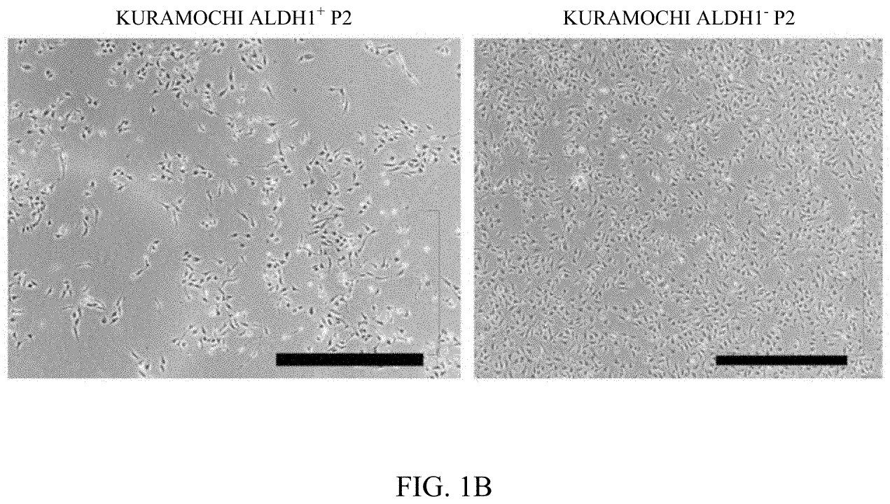

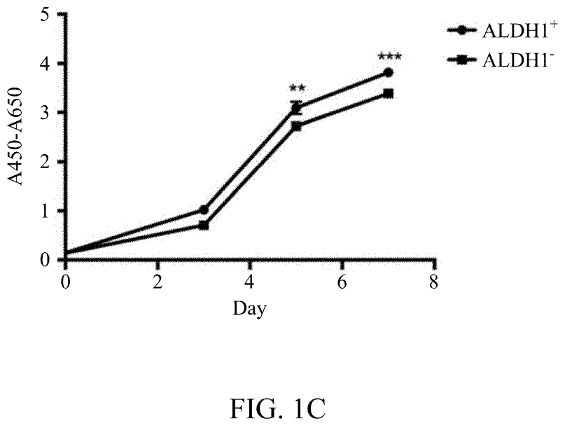

The kuramochi cells were known to exhibit stem cell phenotype. It was found that ALDH⁺ cells consist of 3.6% of the total cell population of kuramochi cells, as shown in FIG. 1A. The morphology of ALDH⁺ and ALDH⁻ kuramochi cells were the same, and all showed cobblestone-like appearance, as shown in FIG. 1B. FIG. 1C shows that the ALDH⁺ cells proliferate faster than the ALDH⁻ cells, reaching a statistically different cell numbers on days 5 to 7 (p<0.01 and p<0.001, respectively). Taken together, these findings show that the ovarian cancer cell line kuramochi cells exhibited stem cell phenotypes. The ALDH⁺ kuramochi cells are considered to be ovarian CSCs.

The half-maximal inhibitory concentration (IC50) of BP is determined in ALDH⁺ and ordinary kuramochi cells. IC50 is determined by assessing the cell viability under different concentration of BP. For example, cell viability was determined by XTT assay (Biological Industries Ltd., Kibbutz Beit Haemek, Israel) following the instruction provided by the manufacturer. Cells were seeded in 96-well plates with a cell density of 2×10³ cells/cm² and treated with different concentrations of BP (0, 15, 30, 60, 120 and 240 μg/ml of BP for ordinary kuramochi cells, and 0, 12.5, 25, 50, 100, 200, and 400 μg/ml BP for ALDH⁺ kuramochi cells) for 48 h. To each 100 μL cell culture in wells of 96-well plates was added with 50 μL XTT/PMS. After 2-5 h of incubation at 37° C., plates were analyzed by spectrophotometry to determine the optical density of the solutions at a wavelength of 450 nm (reference wavelength, 650 nm) to determine viable cell number. The IC50 of both cell types were obtained by the 4PL method described in the previous literature (Sebaugh, J. L. Pharm. Stat. 2011, 10, 128-134). The equation is expressed as follows: Y=d+(a−d)/(1+(X/c)\hat{ }B), where Y is the response, and X is the concentration. The variable “a” is the bottom of the curve, and “d” is the top of the curve. The variable “b” is the slope factor, and “c” is the concentration corresponding to the response midway between “a” and “d” (Chang, Y.-H.; Liu, H.-W.; Chu, T.-Y; Wen, Y-T.; Tsai, R.-K.; Ding, D.-C. Cell Transplant. 2017, 26, 1077-1087). The XTT solutions and N-methyl dibenzopyrazine methyl sulfate (PMS) were defrosted immediately in a water bath at 37° C.

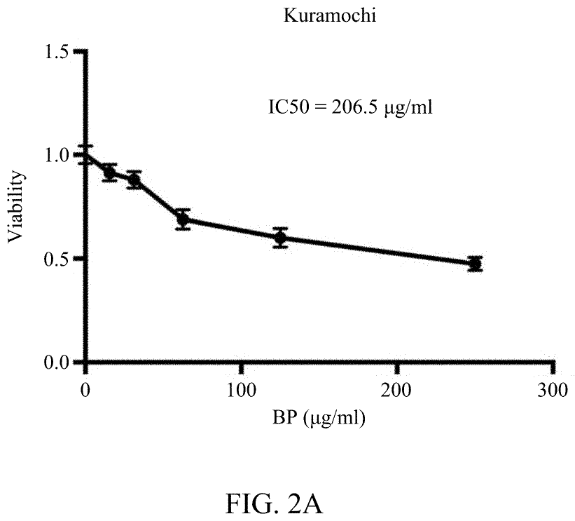

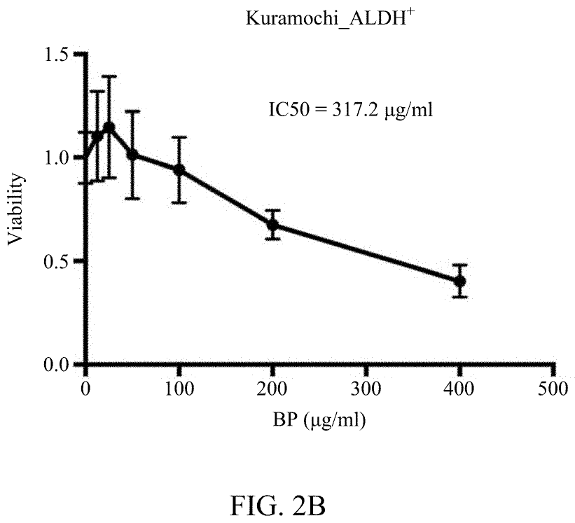

The IC50 of BP for ordinary kuramochi cells after 48 h of treatment was calculated as 206.5 μg/ml (FIG. 2A), while that for the ALDH⁺ kuramochi cells was 317.2 μg/ml (FIG. 2B). This result indicates that higher doses of BP are required to kill kuramochi CSCs.

### Example 2: BP Inhibited ALDH⁺ Kuramochi Cell Migration and Invasion

The ability of ALDH⁺ kuramochi cells to migrate and invade with BP treatment was evaluated.

For assessment of cell migration, kuramochi cells (5×10⁴ cells) were seeded into the top well of a Boyden chamber (24-well transwell) with a pore size of 8 m (Costar, Corning Inc., Corning, N.Y., USA). The cells were allowed to migrate toward the lower well filled with the same culture medium with BP at a concentration of 100 or 200 μg/ml or without BP treatment. After 48 hours of migration, crystal violet (Sigma) was used to stain the migrated cells. The stained cells were counted using a bright-field microscope. Each experiment was repeated three times.

For assessment of cell invasion, the invasion assays were carried out in Matrigel-coated Boyden chambers (filter pore size, 8 μM) in 24-well plates (BD). In the top wells, culture medium without serum was seeded with 5×10⁴ cells. The bottom wells were added with medium containing 10% FBS and BP (0, 100, or 200 μg/ml). Treatments were added to both upper and lower chambers as indicated. After 24 hours, the free cells were removed gently with a cotton swab. The invading cells were fixed with 4% formaldehyde and Giemsa stained. The slides were air-dried and photographed, and the cells were counted.

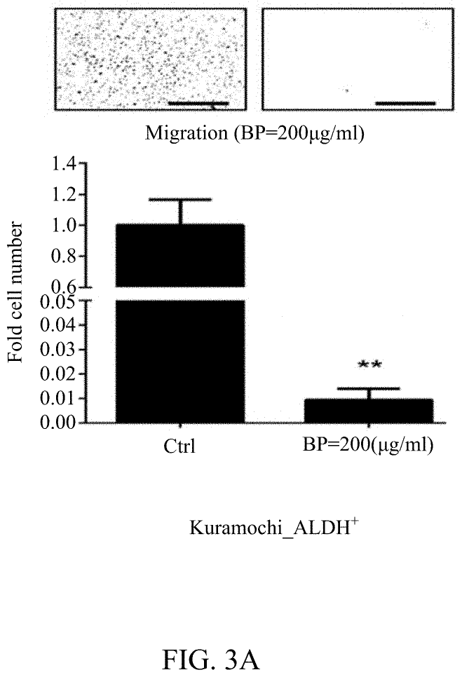

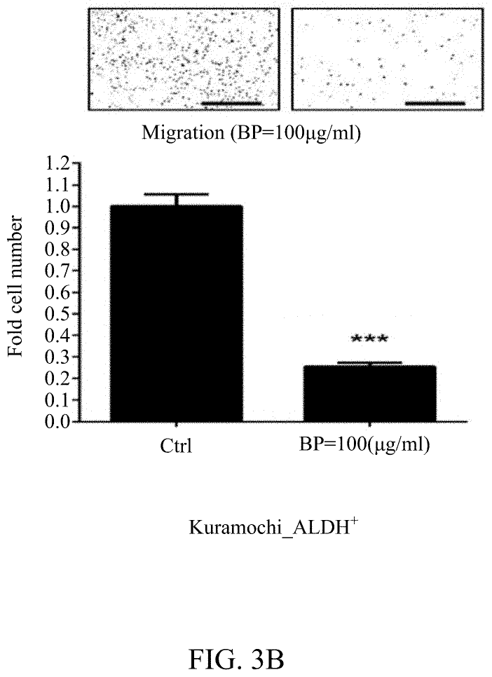

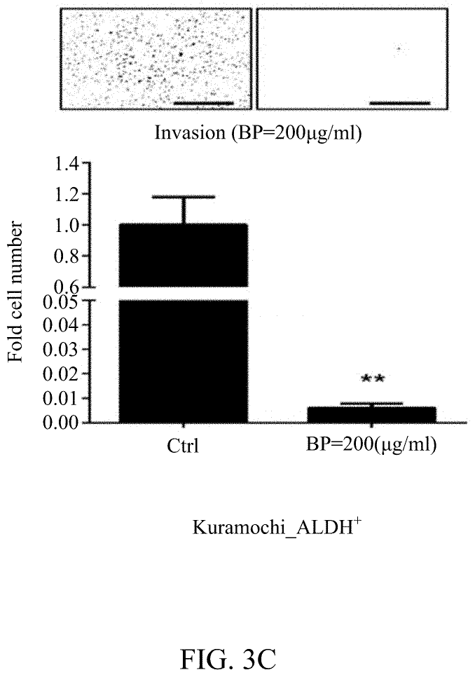

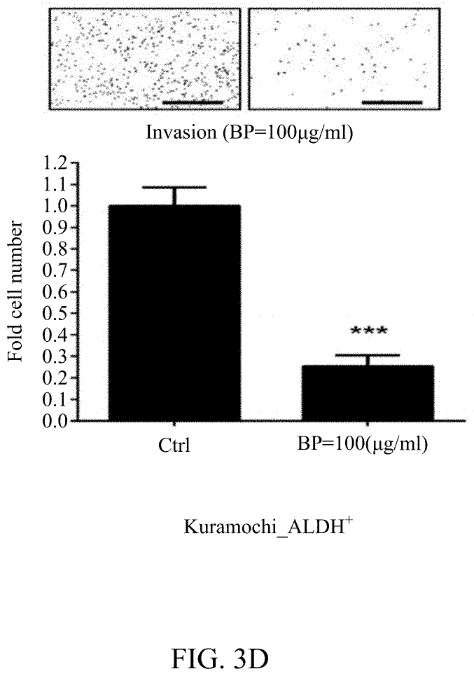

FIGS. 3A and 3B showed cell migration results with BP treatment at concentrations of 200 g/ml and 100 μg/ml, respectively. FIGS. 3C and 3D showed cell invasion results with BP treatment at concentrations of 200 μg/ml and 100 μg/ml, respectively. At both concentrations, BP treatment significantly decreased the migration and invasion of ALDH⁺ cells (100 μg/ml, p<0.01; 200 μg/ml, p<0.001) as compared to untreated cells. These results indicate that BP inhibits typical CSC activities of HGSOC.

### Example 3: BP Activated an Apoptosis Signaling Pathway and Inhibited ALDH⁺ Kuramochi Cell Proliferation Via Apoptosis

The level of apoptosis was compared between ALDH⁺ kuramochi cells with and without BP treatment using the terminal deoxynucleotidyl transferase dUTP nick end labeling (TUNEL) assay. TUNEL assay was carried out to evaluate cell apoptosis using a TUNEL Assay Kit (Roche, Ind., USA) according to the manufacturer's instructions. Specifically, ALDH⁺ kuramochi cells were seeded with 1×10⁵ cells in each well of 12-well culture plates. Cultured cells were allowed to attach for 24 hours, and then treated with 200 μg/ml of BP for 48 hours. Adherent tumor cells were fixed in 4% paraformaldehyde. TUNEL probes were used to detect breaks in DNA strands, followed by incubation in a permeabilization solution for 2 min on ice. Cells were washed twice in phosphate buffered saline (PBS) and the TUNEL reaction mixture was added, followed by incubation at 37° C. for 60 min in a humidified atmosphere in the dark. Samples were washed in PBS twice and observed under a fluorescence microscope.

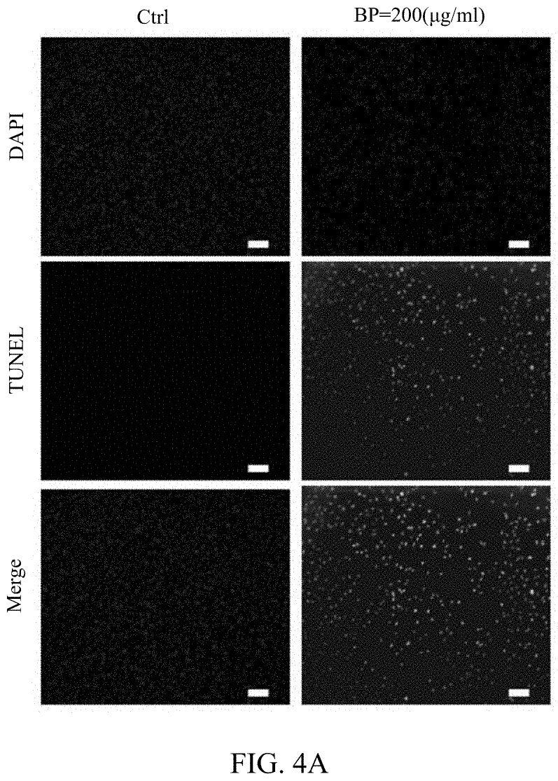

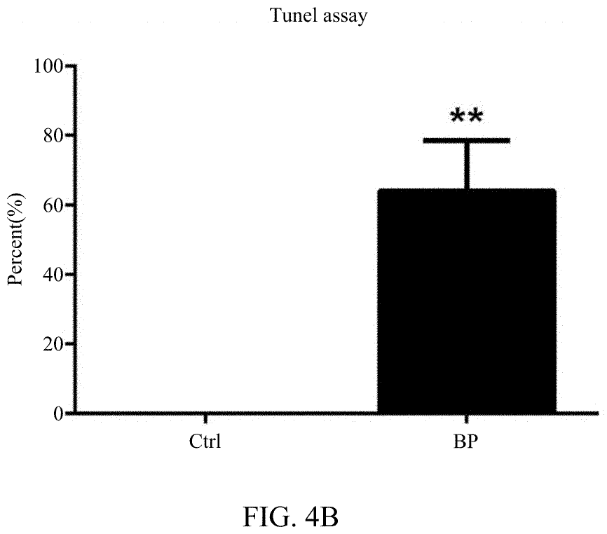

Significantly more TUNEL⁺ cells were observed with BP treatment as shown in FIGS. 4A and 4B (p<0.01). These results indicate that BP treatment of ovarian CSC induces cell apoptosis.

It is known that apoptosis is a targeted cell death program regulated by the caspase protein cascade to prevent inflammatory reactions and damage to surrounding cells (McIlwain, D. R.; Berger, T.; Mak, T. W. Cold Spring Harb. Perspect. Biol. 2013, 5, a008656). The initiator caspases (caspases 8 and 9) activate executioner caspases (caspases 3, 6 and 7) to mediate subsequent reactions that activate the expression of key catabolic proteins and enzymes. Apoptosis occurs via extrinsic and intrinsic pathways. The extrinsic pathway involves signaling from a ligand to a death receptor, which subsequently activates caspase 8, resulting in the activation of downstream executioner caspases (Nair, P.; Lu, M.; Petersen, S.; Ashkenazi, A. Methods Enzymol. 2014, 544, 99-128). Through cleavage of the pro-apoptotic Bid protein, caspase 8 also activates the intrinsic pathway to induce cell death. Cellular stress activates the intrinsic pathway and then activates mitochondrial cytochrome C to activate caspase 9 (Cui, Y; Lu, P.; Song, G.; Liu, Q.; Zhu, D.; Liu, X. Food Chem. Toxicol. 2016, 92, 26-37). Caspase 9 then induces the expression of executioner caspases.

Accordingly, the effect of BP on the apoptotic signaling pathway was further investigated by quantifying the amount of activated caspase proteins by western blot analysis. Specifically, ALDH⁺ kuramochi cells were treated with BP (at 100 μg/ml for 48 hours) and lysed. The cell lysates were loaded onto a gradient 5-20% sodium dodecyl sulfate-polyacrylamide gradient gel. After electrophoretic separation, the proteins were transferred to a polyvinylidene difluoride membrane (Bio-Rad). The membrane was blocked at room temperature in a solution of 3% non-fat dry milk in PBS and 0.1% Tween-20 and then rinsed in PBS/0.1% Tween-20. Blots were incubated with diluted solutions of polyclonal anti-caspase 3 and 8, or anti-cleaved caspase 3, 7, 8, or 9 antibodies (1:200, St. John's Lab, London, UK) and treated with 1:5000 diluted anti-rabbit immunoglobulin G horseradish peroxidase (HRP) for staining (Amersham GE, Taipei, Taiwan). Beta-actin and tubulin proteins (Santa Cruz Biotechnology, Santa Cruz, Calif., USA) were used as internal controls. HRP signals were detected using an electrochemiluminescence kit (Promega, Fitchburg, Wis., USA).

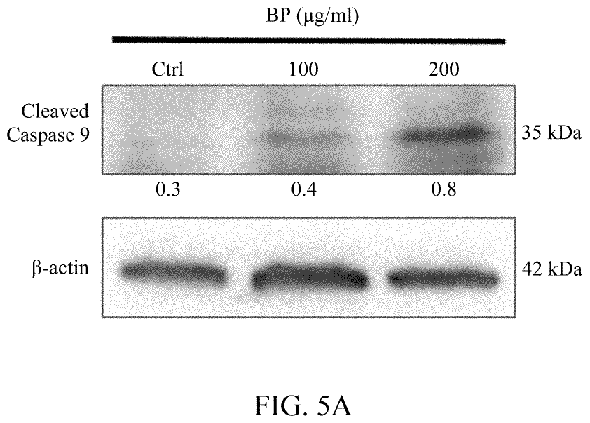

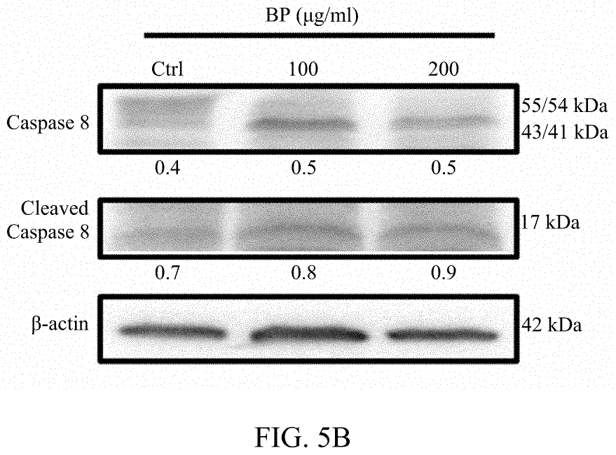

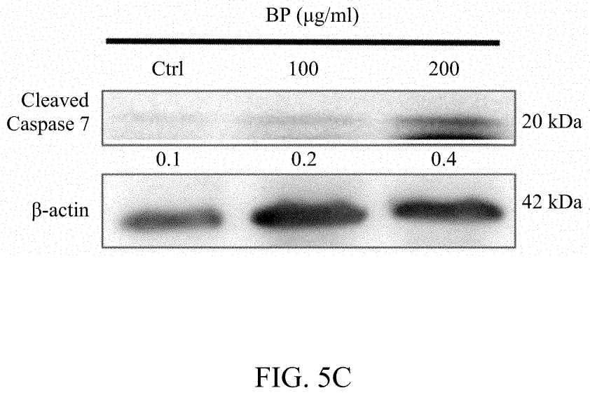

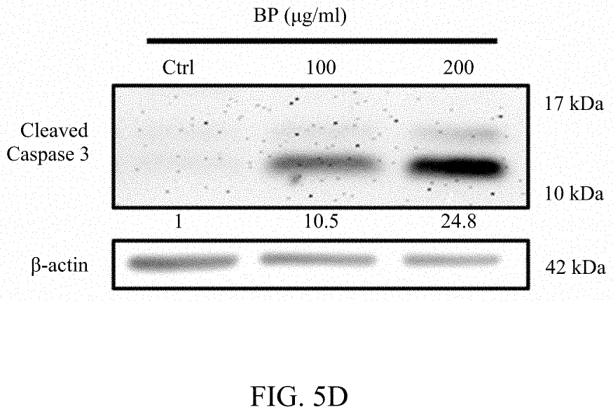

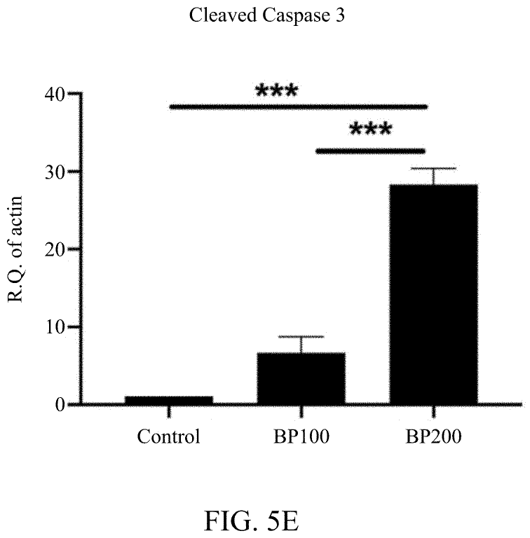

As shown in FIGS. 5A to 5E, BP treatment (at 100 μg/ml or 200 μg/ml) of ALDH⁺ kuramochi cells for 48 hours increased the protein levels of cleaved caspase 9 (FIG. 5A), cleaved caspase 8 (FIG. 5B), cleaved caspase 7 (FIG. 5C), and cleaved caspase 3 (p<0.001, FIGS. 5D and 5E). These results indicate that BP activates both intrinsic and extrinsic apoptosis pathways.

### Example 4: BP Increased the Toxicity of Cisplatin and Paclitaxel

Cisplatin and paclitaxel are standard treatments in adjuvant chemotherapy for ovarian cancer. Paclitaxel stabilizes microtubules, resulting in mitosis arrest and cell death. Cisplatin is an alkylating agent that kills tumor cells. However, both of these drugs are highly toxic and can cause severe conditions such as urticaria, angioedema, and hypertension. Thus, some patients could not complete the complete cycle of chemotherapy because of severe toxicity (Yanaranop, M.; Chaithongwongwatthana, S. Asia Pac. J. Clin. Oncol. 2016, 12, 289-299).

To determine whether BP affects the toxicity of these chemotherapeutic agents, BP was added to cisplatin and paclitaxel treatment of ovarian cancer cells in vitro. ALDH⁺ kuramochi cells (2500 ALDH⁺ cells/well) in a 96-well plate were treated with BP (25 μg/ml) in combination with cisplatin (0, 5, or 10 μM) or with paclitaxel (0, 10 or 50 nM). Each experiment was conducted in triplicate. Plates were incubated at 37° C. with 5% CO₂ for 72 hours. Viable cells were counted by the XTT assay as describe above.

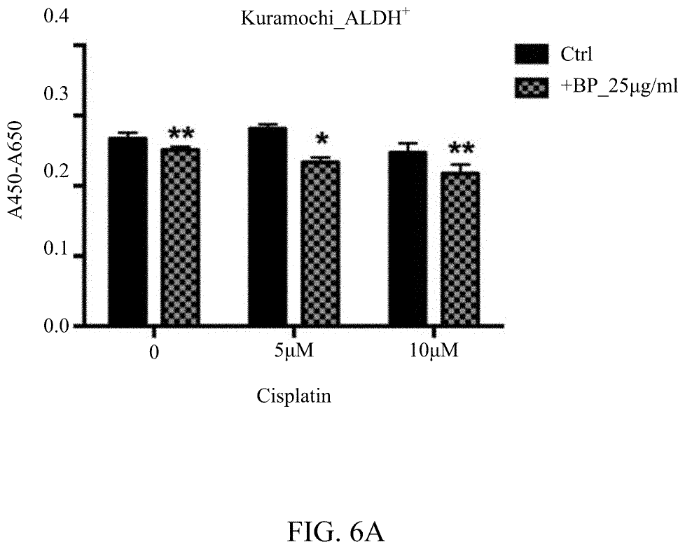

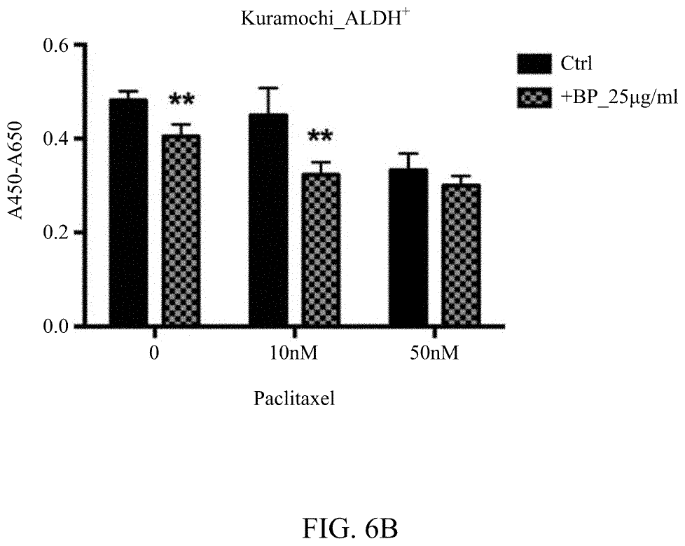

The results show that BP treatment led to significantly lowered cell viability than that of controls (cisplatin without BP) (5 μM, p<0.05; 0 and 10 μM, p<0.01), as shown in FIG. 6A.

Similar effect of BP (25 μg/ml) is observed in combination with paclitaxel treatment (0, 10 or 50 nM). Each paclitaxel treatment is carried out without added BP as control. As shown in FIG. 6B, with zero or low concentration of paclitaxel treatment, the numbers of viable cells treated with additional BP were significantly less than that of control cells (p<0.01).

These results indicate that BP increases the killing effect of cisplatin and paclitaxel on ovarian CSCs. This finding demonstrates that BP can be useful for sensitizing target cells to chemotherapeutic drugs, such that same killing effect can be achieved with less dosage of the chemotherapeutic drugs. Chemotherapeutic treatment of HGSC in combination of BP can thereby decrease their side effects.

### Example 5: BP Inhibited Xenograft Tumor Growth Via Apoptosis

To investigate tumor growth in vivo, the subcutaneous xenograft model is used widely in the research and allows easy detection of tumor growth and size. Herein, human ovarian cancer xenografts were used to investigate the tumor-inhibiting effect of BP in mice. The animal experiment procedures were approved by the Animal Research and Care Committee of the Buddhist Tzu Chi General Hospital (106-48). All procedures were performed in compliance with the National Institutes Health Guide for the Care and Use of Laboratory Animals. Non-obese, diabetic-severe combined immune deficiency mice (NOD-SCID) (strain NOD.CB17-Prkdcscid/JTcu) purchased from Tzu Chi University were used for these experiments.

ALDH⁺ kuramochi cells (1×10⁶) were injected subcutaneously into the backs of 4 to 5-week-old female mice. After tumors had grown to a volume of 50 mm³, mice were separated into two groups (control and treatment groups; n=6 in each group). Controls were treated with vehicle alone (10% dimethyl sulfoxide (DMSO), 20% ethanol in PBS), and the experimental group was treated with different doses of BP (100 or 200 mg/kg) for 5 days. Tumor dimensions (length and width) were measured with calipers, and the tumor volume was determined using the following formula: volume=½ (length×width²). For histological examination, tumor tissues were fixed in 4% paraformaldehyde. Tumors were cut into 6-μm thick sections and stained with hematoxylin and eosin (H&E staining). Tumor tissues were then observed under a microscope at 200× magnification. The morphology and cell density were observed and recorded.

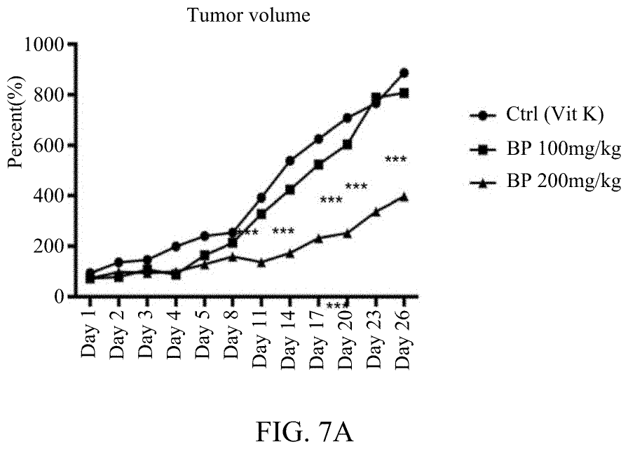

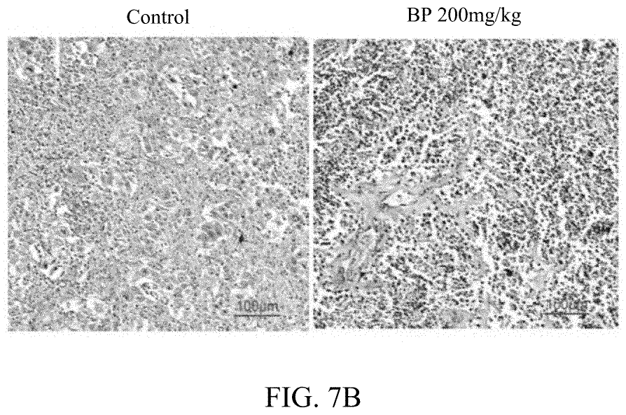

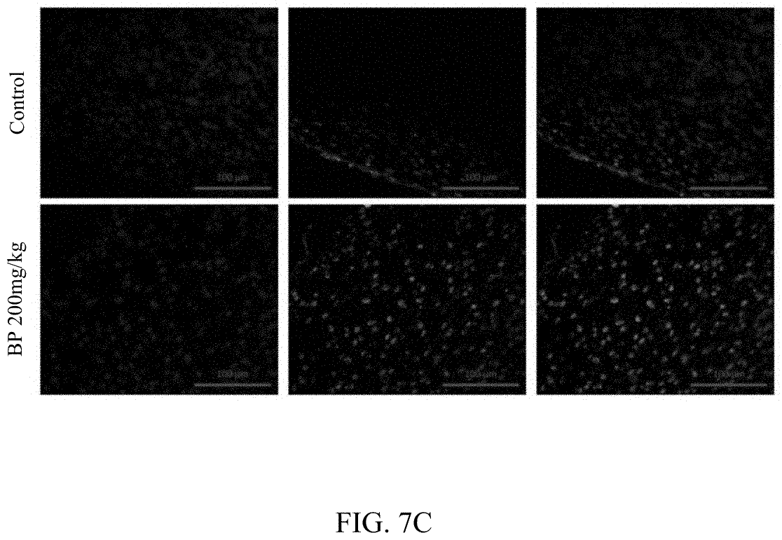

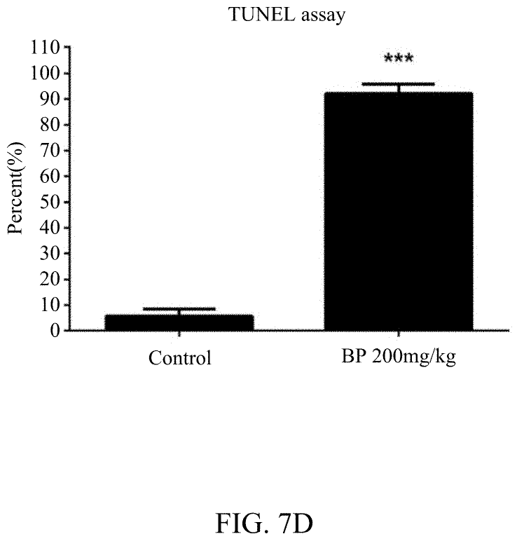

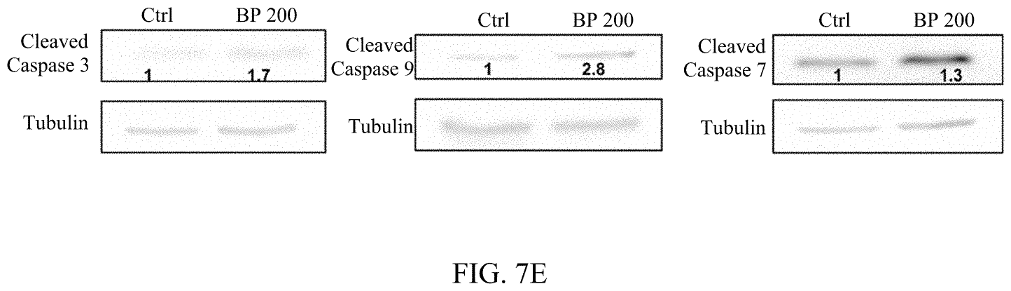

With BP treatment (200 mg/kg), the tumor volumes were smaller than those of control mice treated with the vehicle starting from day 11 to day 26 (p<0.001), as shown in FIG. 7A. H & E staining revealed that the nuclei of BP-treated xenograft tumors were denser compared to those in the control group, shown in FIG. 7B. TUNEL assay was also carried out to evaluate cell viability in the xenograft tumors, using the reagents and procedures as described above but with tumor tissue fixed in formalin and embedded in paraffin and sectioned with a 3 m of thickness. The result showed significantly more TUNEL⁺ cells observed after BP (200 mg/kg) treatment (p<0.001) as shown in FIGS. 7C and 7D. Also, protein levels of cleaved caspase 3, caspase 9, and caspase 7 were examined using western blots as described above to examine apoptotic pathway activation in xenograft tumors. As shown in FIG. 7E, the caspase proteins from ALDH⁺ kuramochi xenograft treated with BP were increased compared to those in the control without BP treatment. Taken together, these results suggest that BP inhibited xenograft growth by activating tumor cell apoptosis.

The foregoing descriptions of the embodiments are only illustrated to disclose the principle and functions of the present disclosure and do not restrict the scope of the present disclosure. It should be understood to those skilled in the art that all modifications and variations according to the principle in the present disclosure should fall within the scope of the appended claims. It is intended that the specification and examples are considered as exemplary only, with a true scope of the disclosure being indicated by the following claims.

What is claimed is:

1. A method for preventing or treating high-grade serous carcinoma in a subject in need thereof, comprising administering a composition comprising an effective amount of n-butylphthalide and a carrier thereof to the subject.

2. The method of claim 1, wherein the subject is provided with at least one additional treatment for the high-grade serous carcinoma.

3. The method of claim 2, wherein the at least one additional treatment comprises surgery, chemotherapy, radiation therapy, targeted therapy, biological therapy or immunotherapy.

4. The method of claim 3, wherein the chemotherapy is an adjuvant chemotherapy.

5. The method of claim 4, wherein the adjuvant chemotherapy comprises administering to the subject an adjuvant chemotherapy drug selected from the group consisting of paclitaxel, carboplatin, cisplatin, gemcitabine, topotecan, and liposomal doxorubucin.

6. The method of claim 1, wherein the high-grade serous carcinoma is a carcinoma originating from a serous epithelial layer in abdominopelvic cavity.

7. The method of claim 1, wherein the high-grade serous carcinoma is high-grade serous ovarian carcinoma, peritoneum serous carcinoma, fallopian tube serous carcinoma or uterine corpus serous carcinoma.

8. The method of claim 1, wherein the high-grade serous carcinoma is high-grade serous ovarian carcinoma.

9. The method of claim 1, wherein the n-butylidenephthalide is purified from natural extracts of plants.

10. The method of claim 9, wherein the n-butylidenephthalide is purified from natural extracts of Angelica sinensis.

11. The method of claim 1, wherein the n-butylidenephthalide is synthetically synthesized.

12. The method of claim 1, wherein the prevention or treatment of the high-grade serous carcinoma comprises inducing apoptosis of cancer stem cells of the high-grade serous carcinoma.

13. The method of claim 12, wherein the induction of the apoptosis increases a protein level of at least one of cleaved caspase 3, cleaved caspase 7, cleaved caspase 8 or cleaved caspase 9.

14. The method of claim 1, wherein the prevention or treatment of the high-grade serous carcinoma comprises inhibition of proliferation of cancer stem cells of the high-grade serous carcinoma.

15. The method of claim 1, wherein the prevention or treatment of the high-grade serous carcinoma comprises inhibition of invasion of cancer stem cells of the high-grade serous carcinoma.

16. The method of claim 1, wherein the prevention or treatment of the high-grade serous carcinoma comprises inhibition of migration of cancer stem cells of the high-grade serous carcinoma.

17. The method of claim 1, wherein the n-butylphthalide is provided at a dosage of from about 10 mg/kg to about 500 mg/kg per day.

18. The method of claim 1, wherein the n-butylphthalide is provided at a dosage of from about 50 mg/kg to about 250 mg/kg per day.

19. The method of claim 1, wherein the subject is a mammal.

20. The method of claim 19, wherein the mammal is a human.
